SEQUENCE LISTING (32 sequences)

SEQ ID NO: 1 - DNA, 816 bp, Gossypium hirsutum
atgggaaggagtccatgttgctccaaggaaggactcaacaaaggagcttggactgctttagaagataaaatacttacatcatatattcatgttcatggtgaaggcaaatggagaaacctccccaagagagctggtttgaagagatgtggcaaaagttgcagacttagatggctgaattatcttagaccagatattaaaagaggcaacatctctcatgatgaagaagaactcattataagactccataatcttcttggcaacagatggtctttaatagctggaaggctacccgggcgaacagacaatgaaatcaagaactactggaacactactttaggtaagagagctaaagctcaagcatccattgaagctaaaacaataccaaccgagtctagactcaatgaaccctcgaaaagttcaaccaaaatcgaagtgattcgaactaaagctattaggtgtagcagcaaggtgatggtcccattacaaccacctgcaactcatcgacatggtcaacatcactgtacaaataataatgaagaaatgggtggtggtattgcaacaattgaagctcacaatggaattcaaatgcttgagtcattgtacagtgatggcggctctgacttgttgagctttgagataaatgaactgttgaaatcacacgatggtggagaatttgaggagaatcctatgcagcagcacttcccgttgggtgaggcagtgtttaaagattggtctactagtcattgtcttgatgacaatggtgccactgatttggaatcattggcctttttgcttgacactgatgaatggccatga

SEQ ID NO: 2 - DNA, 909 bp, Gossypium hirsutum
atgggaaggagtccttgttgttcgaaggaaggccttaacagaggagcttggactgctcttgaagacaaatttcttacagattatatcaaagtacacggtgaaggtcgttggagaaatctccccaaaagagctgggcttaagagatgtgggaaaagttgtaggcttcggtggttgaattatttgagacctgatattaaaagaggtaacatatctcctgacgaggaagagcttatcatcaaactccacaaactcttgggaaacagatggtctttgatagctgggaggcttccagggcgaacagacaatgaaataaagaattactggaacaccaacttaagtaaaagagtttccgatcgtcaaaagtcacccgccgctccttcgaaaaaacccgaggcggctcgacggggaactgctggtaatggcaatgctaatggtaatggtagtggtagttcctcgacacacgtggtgcggacaagggcgacaaggtgctccaaggttttcataaaccctcatcaccacacacaaaacagagacccaaagccttcctcaacttgttcaaatcatggggatcacggggaatctaaaacaatgaatgagttgttattaccgataatgtctgaatccgagaatgaagggacgaccgatcatatatcatcggattttacatttgacttcaacatgggagagttttgtttatcggatcttttgaattccgatttctgcgatgtaagcgagcttaactacagcaaaggttttgattcgtcaccctcaccggatcagcctcctctggatttctccgacgaaatgctaaaagagtggacggccgccgcctccactcactgctctcaccaaagtgtggcttccaatctccagtccttgcctccatttattgaaaatggaattgaatga

SEQ ID NO: 3 - DNA, 915 bp, Glycine max
atggggagaagcccttgttgttcaaaggagggtttgaatagaggtgcttggacagctcatgaagacaaaatcctcagagaatacattagagtccatggtgaaggaagatggagaaaccttcccaaaagagcaggtttgaaaagatgtggaaaaagttgcagacttagatggttgaattatctcagaccagatattaagagaggcaatatatccccagatgaagaagagctcatcatcaggctacacaagctcctgggaaacagatggtctttaatagctgggaggcttccaggacgaacagacaatgaaataaagaattattggaacaccaatttaggaaaaaaggtgaaagatggccaccaaaccactgcaaacaacacacagaatccaatgccccatttggcccccattcatatggccacttcctcaatctccctatctcctcctaaactagattctcgtgttgtccgtacaaaggctaccaagtgctccaaggtattattcctaaacccaccccctcacccatcaatgccgaacaagtccaagactgaggcagaggcagaagcaaggcttgtggatggtgtaattagtaaccaaatggaacacactacatacgacaatgggttcctgtcgtttccagacgaagaaaaagaactctcaacagattttctcatagatttcaacgtgggggatgtttgcttgtctgatctactcaactcagatttctcaaacacgtacaatttcagctgcactaatgttaaccaccacgagcagttatcgccttgttcggaccaacctgatgccatgttctcagatgaagttctcaaggactggacacacagcaattttgcagacgaagcgaatgcttccaacaaccttcattctttcatttcgtttgagtctactgaggaatga

SEQ ID NO: 4 - DNA, 924 bp, Glycine max
atggggagaagcccttgttgttcaaaggagggtttgaatagaggtgcttggacagctcatgaagacaaaatcctgagagaatatattagagtccatggtgaaggaagatggagaaaccttcccaaaagagcaggtttgaaaagatgcgggaaaagttgtagacttcgatggttgaattatctcagaccagatattaagagaggcaatatatccccagatgaagaagagctcatcatcaggctccacaagctcctcggaaacagatggtctttaatagctgggaggcttccaggacgaacagacaacgaaataaagaattattggaacaccaatttagggaaaaaggtgaaagatggtcaccaaacaaccactggaaacaacacacagaatccaatgcccaacccatcaccctccctatctcctcctaaactagattcccatgttgtccgtacaaaggctaccaagtgctccaagttgttattcctaaacccaccccctcacccatcaatgcagaacaagttcaagactgaggcagaagaagaagaagaagaagcaaggcttgtgaatggtgtaattagaaaccaaatggaacacactacatacgacaacgggttcctgtcgtttccggatgaagaaaaagaactctcaacagatttgctcatagatttcaacgtgggggatttctgcttgtctgatcttctcaactcagatttctcaaactcgtacaatttcagctgcaatattaataatgttaacaaccacgagcagttatcgccttgttcggaccaacctgatcctatgttctctgatgaagttctcaaggactggacacacaacaattttgcagacgaagcgaatgcttccaacaatcttcgttctttcatttcgttccttgagtccactgaggaaagattaggagaatga

SEQ ID NO: 5 - DNA, 25 bp, Artificial Sequence
  Primer GhMYB36F
caccatgggaaggagtccatgttgc

SEQ ID NO: 6 - DNA, 22 bp, Artificial Sequence
  Primer GhMYB36R
tcatggccattcatcagtgtca

SEQ ID NO: 7 - DNA, 26 bp, Artificial Sequence
  Primer GhMYB10F
caccatgggaaggagtccttgttgtt

SEQ ID NO: 8 - DNA, 29 bp, Artificial Sequence
  Primer GhMYB10R
tcattcaattccattttcaataaatggag

SEQ ID NO: 9 - DNA, 22 bp, Artificial Sequence
  Primer GhMYB36qRTF
tagcagcaaggtgatggtccca

SEQ ID NO: 10 - DNA, 23 bp, Artificial Sequence
  Primer GhMYB36qRTR
ccgccatcactgtacaatgactc

SEQ ID NO: 11 - DNA, 23 bp, Artificial Sequence
  Primer GhMYB10qRTF
caccacacacaaaacagagaccc

SEQ ID NO: 12 - DNA, 25 bp, Artificial Sequence
  Primer GhMYB10qRTR
tccgatgatatatgatcggtcgtcc

SEQ ID NO: 13 - DNA, 21 bp, Artificial Sequence
  Primer GhHisqRTF
ccgtaccaagcaaaccgcccg

SEQ ID NO: 14 - DNA, 23 bp, Artificial Sequence
  Primer GhHisqRTR
ccaggacggtatcgatgaggctt

SEQ ID NO: 15 - DNA, 20 bp, Artificial Sequence
  Primer EF1aF
aggtccaccaaccttgactg

SEQ ID NO: 16 - DNA, 20 bp, Artificial Sequence
  Primer EF1aR
gagactcgtggtgcatctca

SEQ ID NO: 17 - DNA, 22 bp, Artificial Sequence
  Primer AtANRF
atggaccagactcttacacaca

SEQ ID NO: 18 - DNA, 28 bp, Artificial Sequence
  Primer AtANRR
ttatttagctttgatcaatccttttgac

SEQ ID NO: 19 - DNA, 26 bp, Artificial Sequence
  Primer GhLAR1proF
cacccatcccaatttgaaatgattga

SEQ ID NO: 20 - DNA, 24 bp, Artificial Sequence
  Primer GhLAR1proR
ggttagcgttattgcaagattaga

SEQ ID NO: 21 - DNA, 27 bp, Artificial Sequence
  Primer GrANR1proF
caccgtagatggaggtggaggagtgtg

SEQ ID NO: 22 - DNA, 27 bp, Artificial Sequence
  Primer GrANR1proR
gcttctgtcttaatctgtgtgtgtgtg

SEQ ID NO: 23 - DNA, 32 bp, Artificial Sequence
  Primer GrLAR1proF
cacccaacgacttagacatcctaacttaacac

SEQ ID NO: 24 - DNA, 30 bp, Artificial Sequence
  Primer GrLAR1proR
ggttagcgttattgcaagattagataaaac

SEQ ID NO: 25 - DNA, 23 bp, Artificial Sequence
  Primer MtANRqRTF
caacttctggtcgatacatttgc

SEQ ID NO: 26 - DNA, 21 bp, Artificial Sequence
  Primer MtANRqRTR
ctgagggtatcgtttgctgag

SEQ ID NO: 27 - DNA, 21 bp, Artificial Sequence
  Primer MtLARqRTF
ccgttggatcaattgcacatc

SEQ ID NO: 28 - DNA, 21 bp, Artificial Sequence
  Primer MtLARqRTR
gtaacagttggtagagggtcg

SEQ ID NO: 29 - DNA, 21 bp, Artificial Sequence
  Primer MtDFRqRTF
gtttcaaagacacttgcggag

SEQ ID NO: 30 - DNA, 21 bp, Artificial Sequence
  Primer MtDFRqRTR
gtgataggagaaagggcagtg

SEQ ID NO: 31 - DNA, 22 bp, Artificial Sequence
  Primer MtTUBqRTF
tttgctcctcttacatcccgtg

SEQ ID NO: 32 - DNA, 21 bp, Artificial Sequence
  Primer MtTUBqRTR
cagcacacatcatgtttttgg

(19) United States
(12) Patent Application Publication
(10) Pub. No.: US 2020/0181632 A1
(43) Pub. Date: Jun. 11, 2020

(54) METHODS FOR ENGINEERING PROANTHOCYANIDINS (PAS) IN PLANTS BY AFFECTING MYB TRANSCRIPTION FACTORS

(71) Applicant: UNIVERSITY OF NORTH TEXAS, Denton, TX (US)

(72) Inventors: Richard A. DIXON, Sulphur, OK (US); Nan LU, Denton, TX (US)

(73) Assignee: University of North Texas, Denton, TX (US)

(21) Appl. No.: 16/476,895

(22) PCT Filed: Jan. 17, 2018
(86) PCT No.: PCT/US2018/013983

§ 371 Date: Jul. 10, 2019

Related U.S. Application Data
(60) Provisional application No. 62/447,701, filed on Jan. 18, 2017.

(51) Int. Cl. C12N 15/82

(52) U.S. Cl. CPC C12N 15/8243

(57) ABSTRACT

The amount of proanthocyanidins (PAs) found in cells of plants can be engineered or adjusted through regulation of transcription factors that affect PA biosynthesis. Increasing expression of genes encoding TT2-type MYB transcription factors, including homologs of AtTT2 of Arabidopsis thaliana such as GHMYB36 or GHMYB IO of cotton plants or GmTT2A or GmTT2B of soybean plants, leads to increased PA content. Expression of these genes can be increased through any suitable methods, including mutation of the genes and transformation of plant cells to include exogenous genes resulting in increased expression.

### BACKGROUND

This application claims priority to U.S. Provisional Patent Application No. 62/447,701, filed Jan. 18, 2017, entitled “Methods for Engineering Proanthocyanidins (PAs) in Plants by Affecting MYB Transcription Factors,” the entire content of which is hereby incorporated by reference.

This disclosure pertains to regulating, adjusting, or engineering the content of extractable proanthocyanidins (PAs) in plants.

Proanthocyanidins (PAs), also known as condensed tannins, are important secondary metabolites involved in stress resistance in plants, and are health supplements that help to reduce cholesterol levels. As one of the most widely grown crops in the world, cotton provides the majority of natural fabrics and is a supplemental food for ruminant animals. Previous studies have suggested that PAs present in cotton are a major contributor to fiber color. However, the biosynthesis of PAs in cotton still remains to be elucidated.

Proanthocyanidins (PAs) are synthesized through the flavonoid biosynthesis pathway. PAs play vital roles in plant defense against pathogens and other diseases. PAs are antioxidants that are used in human health supplements, and they have been suggested to possess anti-cancer activity. As phenolic compounds, PAs also provide astringency in beverages such as green tea and red wine. In some plants like Arabidopsis, soybean and Medicago, PAs exclusively accumulate in seeds, while in other species like cotton and poplar they accumulate in various tissues, including seed coat, leaf, bark and. Studies have suggested that PAs in feed can help prevent digestive disorders in ruminant animals, due to the fact that polymeric PAs can bind to proteins and slow down their rate of fermentation.

In plants, biosynthesis of PAs and anthocyanins share many enzymes such as chalcone synthase (CHS), chalcone isomerase (CHI), dihydroflavonol 4-reductase (DFR), and anthocyanidin synthase (ANS). Two key structural enzymes unique to the PA biosynthesis pathway are anthocyanidin reductase (ANR) and leucoanthocyanidin reductase (LAR). Biochemical analysis shows that ANR converts anthocyanidins (cyanidin, delphinidin and pelargonidin) to the corresponding flavan 3-ols (catechin/epicatechin, gallocatechin/epigallocatechin, afzelechin/epiafzelechin), whereas LAR can reduce leucocyanidin to catechin, although it has recently been shown to possess an alternative function in regulating PA oligomerization. FIG. 1 shows the general flavonoid pathway in plants. F3H is flavanone 3 hydroxylase; F3′H is flavonoid 3′ hydroxylase; FLS is flavonol synthase; and UGT is UDP glucosyltransferase. Catechin, epicatechin and other flavan 3-ols are considered building blocks for PA biosynthesis. In Arabidopsis, where knockout of AtANR leads to predictable accumulation of anthocyanins in seed coats, no LAR homologs are present. Other proteins such as UGT72L1 and MATE1 are proposed to be involved in the glycosylation and transport of PA monomers, respectively, followed by subsequent polymerization in vacuoles.

Regulation of the PA biosynthesis pathway has generally been agreed to involve R2R3-type MYB, WD40 and bHLH (basic Helix-Loop-Helix) transcription factors. These proteins together form a ternary complex (Myb-bHLH-WD40, or MBW), which binds to promoters of structural genes, especially ANR and LAR, and enhances their expression levels. Among the three proteins in the complex, MYB is believed to be the core member. Recently it has been discovered that several repressors are also involved in the regulation process. It would appear that besides the R2R3 domains at the N-terminal end, these repressor proteins also share conserved motifs near the C-terminal end. In Arabidopsis, the positive MYB regulator is known as TT2, and knockout of this gene leads to loss of PAs in seed coats which results in a transparent testa (tt) phenotype. Expression of AtTT2 and AtANR follow the same pattern, reaching highest transcript levels around the globular stage of seed development. Homologs of AtTT2 from other plant species have been reported, including from Medicago truncatula, Vitis vinefera, Lotus japonicus and Trifolium arvense. In L. japonicus, more than one homolog of AtTT2 exists (LjTT2a, LjTT2b and LjTT2c), although the three genes have different expression profiles and have different abilities for binding with other proteins to form the ternary complex.

Non-TT2 like MYB transcription factors might also be involved in regulating PA biosynthesis in plants; these include AtMYB5, VvMYBPA1 and DkMYB4, and they reside in a different clade based on sequence analysis in phylogenetic studies. Genes in this clade share a conserved motif different from the TT2 type domain near the C-terminal end and might play additional functional roles such as in trichome development and mucilage accumulation. Bimolecular fluorescence complementation (BiFC) analysis suggested physical interactions between MtMYB5 (homolog of AtMYB5) and MtMYB14 (homolog of AtTT2), and these transcription factors function synergistically in PA regulation in Medicago.

Cultivated cotton (Gossypium hirsutum) is the world's most important provider of fiber products. Besides, cotton is also a good source of oil and protein. Cottonseed meal can supply PAs when mixed in animal feed. Evolutionarily, cultivated tetraploid upland cotton (Gossypium hirsutum, AADD) is the result of hybridization between its two diploid ancestors, Gossypium arboreum (AA genome) and Gossypium raimondii (DD genome). Unlike Arabidopsis, cotton accumulates PAs not only in the seed coat, but also in leaf, stem, fiber and root. Recent studies have found that PA content in cotton is related to fiber color. Several transcriptome studies have been published exploring gene expression profiles in brown fiber cotton and cottonseeds in the context of breeding naturally colored fibers. In these cases, it was obvious that expression patterns of structural genes such as GhANR, GhLAR and GhDFR all show significant differences between white and brown cotton fibers. The GhANR gene product has recently been biochemically characterized, and loss of function of GhANR in cotton leads to accumulation of anthocyanins in leaf and stem due to accumulation and glycosylation of anthocyanidin substrate, consistent with previous results with the Arabidopsis banyuls mutant. The newly released tetraploid cotton draft genome sequences provide new resources for future research in breeding and genetic engineering of cotton.

Engineering PAs in plants has been of considerable interest in recent years, especially for forage legumes. Success has been obtained in tobacco where PAP1 (Production of Anthocyanin Pigment 1) and MtANR genes were co-transformed, and in Trifolium where TaMYB14 was ectopically expressed, as well as in Lotus and apple where maize Sn and Lc transcription factors were used, respectively. Sn and Lc, which are bHLH family transcription factors from maize, have been considered to only regulate anthocyanin biosynthesis and not PAs.

### SUMMARY

The present disclosure relates generally to adjusting the amount of proanthocyanidins (PAs) in plants through regulation of transcription factors that affect PA biosynthesis.

AtTT2 (transparent testa 2) is a MYB family transcription factor from Arabidopsis that initiates the biosynthesis of PAs by inducing the expression of multiple genes in the pathway. Broadly, genes for MYB family transcription factors may be targeted and affected in order to regulate the PA biosynthesis pathway in cotton plants, soybean plants, alfalfa plants, and other plants. As discussed below, two R2R3-type MYB transcription factors from G. hirsutum were isolated that are homologous to AtTT2. Expression analysis showed both genes were expressed at different levels in various cotton tissues, including leaf, seed coat and fiber. Protoplast transactivation assays revealed that these two GhMYBs were able to activate promoters of genes encoding enzymes in the PA biosynthesis pathway, including anthocyanidin reductase (ANR) and leucoanthocyanidin reductase (LAR). Complementation experiments showed that both of the GhMYBs were able to recover the transparent testa seed coat phenotype of the Arabidopsis tt2 mutant by restoring PA biosynthesis. Ectopic expression of either of the two GhMYBs in Medicago truncatula hairy roots increased the contents of anthocyanins and PAs compared to control lines, and transcript levels of PA biosynthetic genes were also elevated in lines expressing GhMYBs.

In addition, soybean MYB genes GmMYBx and GmMYBy are soybean MYB genes encoding TT2-type MYB transcription factors that have sequences similar to the two GhMYBs. These and other genes for MYB family transcription factors can be affected, particularly in ways that enhance expression of the genes, in order to increase PA contents in various types of plants.

PAs are important plant specialized metabolites that accumulate in seed coats, leaves and roots. PA biosynthesis pathways in plants have been well studied over the past decades, and both positive and negative regulators have been revealed in a number of species. These transcription factors control the temporal and spatial distribution of PAs by regulating expression patterns of key enzymes in the pathway, especially ANR and LAR. In Arabidopsis, characterization of a collection of transparent testa mutants led to the discovery of the AtTT2 gene, which is a positive regulator of PA biosynthesis. Later it was found that families of related R2R3 MYB transcription factors with shared conserved functional domains exist in many species. Two homologs of AtTT2 from tetraploid cotton are identified herein. These were able to complement the mutant phenotype of Arabidopsis tt2 and induce PA accumulation in Medicago hairy root cultures. Compared to Arabidopsis wild type and tt2 mutant, the GHMYB36 overexpres sing lines accumulated high concentration of PAs in the roots. Transcript analysis showed that GHMYB36 and GHMYB10 had different tissue-specific expression profiles, with relatively higher levels in leaves compared to seed coats. Transactivation assays indicated that GHMYB36 and GHMYB10 function as part of a ternary complex with a WD40 and bHLH protein for transactivation of PA pathways genes, and that the complex with both MYBs is more effective than that with a single MYB. This supports recent findings suggesting similar enhanced responses to pairs of MYBs in a “quaternary complex” for PA pathway activation in Medicago.

Transactivation data using TaMYB14 and MtMYB14 suggest that one LAR (LAR1) gene is the main target of MYB regulation during PA biosynthesis in cotton. In a transcriptome analysis using brown and whiter fiber cotton, the expression level of the LAR1 gene was much higher than that of LAR2. Similarly, in brown soybean, which produces epicatechin-based PAs, there are two ANR genes that have been biochemically characterized, but only GmANR1 expression correlates with the PA accumulation pattern. The exact functions of cotton LAR2 or soybean ANR2 remain to be determined.

The composition of PAs, including the ratios of the different monomeric flavan 3-ols and their degree of polymerization, may influence their functions as important specialized metabolites and health supplements. To evaluate the full potential of the cotton MYBs for biotechnological application, it was important to test whether the composition of PAs induced by ectopic expression of GHMYB36 and GHMYB10 in Arabidopsis and Medicago had been altered. PAs in both Arabidopsis and Medicago are epicatechin-based, while in cotton the major building blocks are gallocatechins and catechins. Results showed that even though MtANR and MtLAR expression levels were activated at different levels in different lines, the PAs accumulated in Medicago hairy root cultures expressing GHMYB36 and GHMYB10 were still epicatechin-based.

In plants, the biosynthesis of (epi)gallocatechins is largely dependent on the existence and functionality of flavonoid 3′,5′-hydroxylases (F3′5′H), which are responsible for hydroxylation of flavonoids on the B-ring resulting in production of delphinidin as one of the anthocyanidins. Some plants such as Arabidopsis lack this gene, resulting in the absence of (epi)gallocatechin in PAs. In plants with functional F3′5′H genes such as cotton, gallocatechin and epigallocatechin can be detected. However, whether this family of genes is directly regulated by TT2-type MYBs is not yet clear, since target genes of TT2s such as ANR and LAR mainly encode the downstream enzymes of the PA biosynthesis pathway. In this case, TT2-type MYBs from cotton did not alter the epicatechin-based PA composition in Medicago hairy roots.

Improving PA concentrations in forage legumes and other crop species has always been a focus in agriculture. As an important economic crop, cotton was one of the first species in which genetic engineering approaches were applied, mainly with Bt and other insect resistance genes. PAs can protect plants against herbivores, so PA engineering could provide an alternative to the Bt gene. The importance of engineering PAs in cotton has been highlighted by the recent discovery that PA concentrations directly impact fiber color.

### BRIEF DESCRIPTION OF THE DRAWINGS

FIG. 1 shows the general flavonoid pathway in plants.

FIG. 2 shows a multiple protein sequence alignment of MYB transcription factors.

FIG. 3 shows a phylogenetic tree of GHMYB36, GHMYB10 and other known R2R3-type MYB transcription factors.

FIG. 4 shows transcript levels of GHMYB36 (a) and GHMYB10 (b) in different tissues of cotton as determined by qRT-PCR.

FIG. 5 shows transactivation assays for GHMYB36 and GHMYB10 with GhLAR (a), GrLAR (b) and GrANR (c) promoters, and (d) constructs used in the assays.

FIG. 6 shows RT-PCR of Arabidopsis housekeeping gene EFla, endogenous AtANR, GHMYB36 and GHMYB10 using leaf samples from wild type, tt2 and transgenic lines.

FIG. 7 shows transcript levels of MtANR, MtLAR and MtDFR in transgenic Medicago hairy root cultures transformed with GUS, GHMYB36 or GHMYB10.

FIG. 8 shows quantification of (a) epicatechin, (b) insoluble PA, and (c) anthocyanin in transgenic Medicago hairy root cultures transformed with GUS, GHMYB36 or GHMYB10.

FIG. 9 shows (a) normal phase HPLC with post-column derivatization showing size distribution of soluble PAs from Medicago hairy root cultures transformed with GUS or GHMYB36, (b) HPLC analysis of phloroglucinolysis products of PAs from Medicago hairy root cultures transformed with GHMYB36 or GUS, and (c) acid butanol hydrolysis of insoluble PAs extracted from GHMYB36 lines.

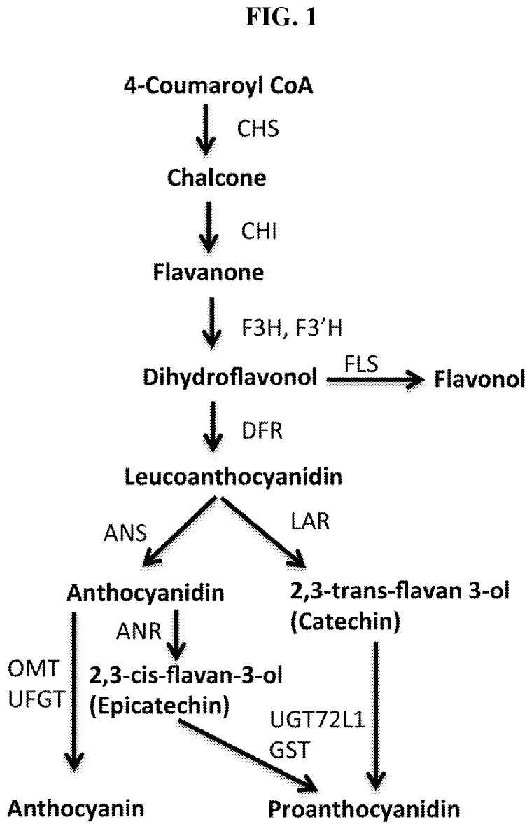

### DETAILED DESCRIPTION OF PREFERRED EMBODIMENTS

Generally, the present disclosure relates to adjusting the amount of proanthocyanidins (PAs) in plants by regulating transcription factors that affect PA biosynthesis. In preferred embodiments, the present disclosure pertains to increasing the amount of PAs found in plants, including cotton plants, soybean plants, and other plants, by increasing the expression of TT2-type MYB transcription factors. Expression of these genes can be increased through any suitable methods, including mutation of the genes and transformation of plant cells to include exogenous genes resulting in increased expression in the cells. In additional preferred embodiments, the TT2-type MYB transcription factors are from G. hirsutum (cotton) and are encoded by the genes GHMYB36 (SEQ ID NO:1) and GHMYB10 (SEQ ID NO:2). In further preferred embodiments, the TT2-type MYB transcription factors are from Glycine max (soybean) and are encoded by the genes GmTT2A (SEQ ID NO:3) and GmTT2B (SEQ ID NO:4). The sequences of these genes are shown below.

GHMYB36:
(SEQ ID NO: 1)
ATGGGAAGGAGTCCATGTTGCTCCAAGGAAGGACTCAACAAAGGAGCTTG
GACTGCTTTAGAAGATAAAATACTTACATCATATATTCATGTTCATGGTG
AAGGCAAATGGAGAAACCTCCCCAAGAGAGCTGGTTTGAAGAGATGTGGC
AAAAGTTGCAGACTTAGATGGCTGAATTATCTTAGACCAGATATTAAAAG
AGGCAACATCTCTCATGATGAAGAAGAACTCATTATAAGACTCCATAATC
TTCTTGGCAACAGATGGTCTTTAATAGCTGGAAGGCTACCCGGGCGAACA
GACAATGAAATCAAGAACTACTGGAACACTACTTTAGGTAAGAGAGCTAA
AGCTCAAGCATCCATTGAAGCTAAAACAATACCAACCGAGTCTAGACTCA
ATGAACCCTCGAAAAGTTCAACCAAAATCGAAGTGATTCGAACTAAAGCT
ATTAGGTGTAGCAGCAAGGTGATGGTCCCATTACAACCACCTGCAACTCA
TCGACATGGTCAACATCACTGTACAAATAATAATGAAGAAATGGGTGGTG
GTATTGCAACAATTGAAGCTCACAATGGAATTCAAATGCTTGAGTCATTG
TACAGTGATGGCGGCTCTGACTTGTTGAGCTTTGAGATAAATGAACTGTT
GAAATCACACGATGGTGGAGAATTTGAGGAGAATCCTATGCAGCAGCACT
TCCCGTTGGGTGAGGCAGTGTTTAAAGATTGGTCTACTAGTCATTGTCTT
GATGACAATGGTGCCACTGATTTGGAATCATTGGCCTTTTTGCTTGACAC
TGATGAATGGCCATGA
GHMYB10:
(SEQ ID NO: 2)
ATGGGAAGGAGTCCTTGTTGTTCGAAGGAAGGCCTTAACAGAGGAGCTTG
GACTGCTCTTGAAGACAAATTTCTTACAGATTATATCAAAGTACACGGTG
AAGGTCGTTGGAGAAATCTCCCCAAAAGAGCTGGGCTTAAGAGATGTGGG
AAAAGTTGTAGGCTTCGGTGGTTGAATTATTTGAGACCTGATATTAAAAG
AGGTAACATATCTCCTGACGAGGAAGAGCTTATCATCAAACTCCACAAAC
TCTTGGGAAACAGATGGTCTTTGATAGCTGGGAGGCTTCCAGGGCGAACA
GACAATGAAATAAAGAATTACTGGAACACCAACTTAAGTAAAAGAGTTTC
CGATCGTCAAAAGTCACCCGCCGCTCCTTCGAAAAAACCCGAGGCGGCTC
GACGGGGAACTGCTGGTAATGGCAATGCTAATGGTAATGGTAGTGGTAGT
TCCTCGACACACGTGGTGCGGACAAGGGCGACAAGGTGCTCCAAGGTTTT
CATAAACCCTCATCACCACACACAAAACAGAGACCCAAAGCCTTCCTCAA
CTTGTTCAAATCATGGGGATCACGGGGAATCTAAAACAATGAATGAGTTG
TTATTACCGATAATGTCTGAATCCGAGAATGAAGGGACGACCGATCATAT
ATCATCGGATTTTACATTTGACTTCAACATGGGAGAGTTTTGTTTATCGG
ATCTTTTGAATTCCGATTTCTGCGATGTAAGCGAGCTTAACTACAGCAAA
GGTTTTGATTCGTCACCCTCACCGGATCAGCCTCCTCTGGATTTCTCCGA
CGAAATGCTAAAAGAGTGGACGGCCGCCGCCTCCACTCACTGCTCTCACC
AAAGTGTGGCTTCCAATCTCCAGTCCTTGCCTCCATTTATTGAAAATGGA
ATTGAATGA
GmTT2A:
(SEQ ID NO: 3)
ATGGGGAGAAGCCCTTGTTGTTCAAAGGAGGGTTTGAATAGAGGTGCTTG
GACAGCTCATGAAGACAAAATCCTCAGAGAATACATTAGAGTCCATGGTG
AAGGAAGATGGAGAAACCTTCCCAAAAGAGCAGGTTTGAAAAGATGTGGA
AAAAGTTGCAGACTTAGATGGTTGAATTATCTCAGACCAGATATTAAGAG
AGGCAATATATCCCCAGATGAAGAAGAGCTCATCATCAGGCTACACAAGC
TCCTGGGAAACAGATGGTCTTTAATAGCTGGGAGGCTTCCAGGACGAACA
GACAATGAAATAAAGAATTATTGGAACACCAATTTAGGAAAAAAGGTGAA
AGATGGCCACCAAACCACTGCAAACAACACACAGAATCCAATGCCCCATT
TGGCCCCCATTCATATGGCCACTTCCTCAATCTCCCTATCTCCTCCTAAA
CTAGATTCTCGTGTTGTCCGTACAAAGGCTACCAAGTGCTCCAAGGTATT
ATTCCTAAACCCACCCCCTCACCCATCAATGCCGAACAAGTCCAAGACTG
AGGCAGAGGCAGAAGCAAGGCTTGTGGATGGTGTAATTAGTAACCAAATG
GAACACACTACATACGACAATGGGTTCCTGTCGTTTCCAGACGAAGAAAA
AGAACTCTCAACAGATTTTCTCATAGATTTCAACGTGGGGGATGTTTGCT
TGTCTGATCTACTCAACTCAGATTTCTCAAACACGTACAATTTCAGCTGC
ACTAATGTTAACCACCACGAGCAGTTATCGCCTTGTTCGGACCAACCTGA
TGCCATGTTCTCAGATGAAGTTCTCAAGGACTGGACACACAGCAATTTTG
CAGACGAAGCGAATGCTTCCAACAACCTTCATTCTTTCATTTCGTTTGAG
TCTACTGAGGAATGA
GmTT2B:
(SEQ ID NO: 4)
ATGGGGAGAAGCCCTTGTTGTTCAAAGGAGGGTTTGAATAGAGGTGCTTG
GACAGCTCATGAAGACAAAATCCTGAGAGAATATATTAGAGTCCATGGTG
AAGGAAGATGGAGAAACCTTCCCAAAAGAGCAGGTTTGAAAAGATGCGGG
AAAAGTTGTAGACTTCGATGGTTGAATTATCTCAGACCAGATATTAAGAG
AGGCAATATATCCCCAGATGAAGAAGAGCTCATCATCAGGCTCCACAAGC
TCCTCGGAAACAGATGGTCTTTAATAGCTGGGAGGCTTCCAGGACGAACA
GACAACGAAATAAAGAATTATTGGAACACCAATTTAGGGAAAAAGGTGAA
AGATGGTCACCAAACAACCACTGGAAACAACACACAGAATCCAATGCCCA
ACCCATCACCCTCCCTATCTCCTCCTAAACTAGATTCCCATGTTGTCCGT
ACAAAGGCTACCAAGTGCTCCAAGTTGTTATTCCTAAACCCACCCCCTCA
CCCATCAATGCAGAACAAGTTCAAGACTGAGGCAGAAGAAGAAGAAGAAG
AAGCAAGGCTTGTGAATGGTGTAATTAGAAACCAAATGGAACACACTACA
TACGACAACGGGTTCCTGTCGTTTCCGGATGAAGAAAAAGAACTCTCAAC
AGATTTGCTCATAGATTTCAACGTGGGGGATTTCTGCTTGTCTGATCTTC
TCAACTCAGATTTCTCAAACTCGTACAATTTCAGCTGCAATATTAATAAT
GTTAACAACCACGAGCAGTTATCGCCTTGTTCGGACCAACCTGATCCTAT
GTTCTCTGATGAAGTTCTCAAGGACTGGACACACAACAATTTTGCAGACG
AAGCGAATGCTTCCAACAATCTTCGTTCTTTCATTTCGTTCCTTGAGTCC
ACTGAGGAAAGATTAGGAGAATGA

In additional preferred embodiments, the present disclosure pertains to a method for producing a modified plant having increased proanthocyanidin (PA) content in cells of the plant compared to an unmodified plant of the same species, comprising the steps of increasing expression of at least one gene encoding a TT2-type MYB transcription factor in the cells of the plant and producing a modified plant having increased expression of TT2-type MYB transcription factors and increased proanthocyanidin (PA) content in cells of the modified plant. In preferred embodiments, the gene encoding a TT2-type MYB transcription factor is a homolog of AtTT2 of Arabidopsis thaliana. In additional preferred embodiments, the plant is a cotton plant and the gene encoding a TT2-type MYB transcription factor is GHMYB36 or GHMYB10. In certain embodiments, GHMYB36 comprises SEQ ID NO:1, and GHMYB10 comprises SEQ ID NO:2. In further preferred embodiments, the plant is a soybean plant and the gene encoding a TT2-type MYB transcription factor is GmTT2A or GmTT2B. In certain embodiments, GmTT2A comprises SEQ ID NO:3, and GmTT2B comprises SEQ ID NO:4.

In preferred embodiments, the step of increasing expression of at least one gene encoding a TT2-type MYB transcription factor includes introducing a mutation into the gene encoding a TT2-type MYB transcription factor in cells of the plant, wherein the mutation results in increased expression of the gene encoding the TT2-type MYB transcription factor.

In additional preferred embodiments, the gene encoding a TT2-type MYB transcription factor is an exogenous gene relative to the plant being modified. In these preferred embodiments, the step of increasing expression of the at least one gene encoding a TT2-type MYB transcription factor includes transforming at least one cell of the plant with the gene encoding a TT2-type MYB transcription factor to produce a modified plant having increased expression of the gene encoding the TT2-type MYB transcription factor. In additional preferred embodiments, the transformed plant is a non-cotton plant and the exogenous gene encoding TT2-type MYB transcription factors is GHMYB36 or GHMYB10. In additional preferred embodiments, the transformed plant is a non-soybean plant and the exogenous gene encoding TT2-type MYB transcription factors is GmTT2A or GmTT2B. In additional preferred embodiments, the transformed plant is a Medicago truncatula or Arabidopsis thaliana plant. In further preferred embodiments, the transformed plant is a cotton plant, a soybean plant, or an alfalfa plant. In further preferred embodiments, the transformed plant may be further transformed with an exogenous gene encoding an additional MYB transcription factor to produce a modified plant having increased expression of the exogenous gene encoding the additional MYB transcription factor, as well as increased expression of the exogenous gene encoding the TT2-type MYB transcription factor.

Further preferred embodiments relate to a modified plant having increased proanthocyanidin (PA) content in cells of the plant compared to an unmodified plant of the same species, wherein cells of the plant have increased expression of at least one gene encoding a TT2-type MYB transcription factor. Additional preferred embodiments include a seed of this modified plant. In further preferred embodiments of the modified plant, the gene encoding a TT2-type MYB transcription factor is a homolog of AtTT2 of Arabidopsis thaliana. In addition, in preferred embodiments, the modified plant is a cotton plant and the gene encoding a TT2-type MYB transcription factors is GHMYB36 or GHMYB10. In these preferred embodiments, GHMYB36 may have SEQ ID NO:1, and GHMYB10 may have SEQ ID NO:2. In further preferred embodiments, the modified plant is a soybean plant and the gene encoding a TT2-type MYB transcription factor is GmTT2A or GmTT2B. In these embodiments, GmTT2A may have SEQ ID NO:3, and GmTT2B may have SEQ ID NO:4.

Additional preferred embodiments include a modified plant in which cells of the plant have a mutation in the gene encoding a TT2-type MYB transcription factor, and the mutation results in increased expression of the gene encoding the TT2-type MYB transcription factor in cells of the modified plant.

In further preferred embodiments of the modified plant, the gene encoding a TT2-type MYB transcription factor is an exogenous gene, and at least one cell of the plant is transformed with the exogenous gene encoding a TT2-type MYB transcription factor to produce a modified plant having increased expression of the gene encoding the TT2-type MYB transcription factor. In additional preferred embodiments, the transformed plant is a non-cotton plant and the exogenous gene encoding TT2-type MYB transcription factors is GHMYB36 or GHMYB10. In additional preferred embodiments, the transformed plant is a non-soybean plant and the exogenous gene encoding TT2-type MYB transcription factors is GmTT2A or GmTT2B. In additional preferred embodiments, the transformed plant is a Medicago truncatula or Arabidopsis thaliana plant. In further preferred embodiments, the transformed plant is a cotton plant, a soybean plant, or an alfalfa plant. In further preferred embodiments, the modified transformed plant may be further transformed with an exogenous gene encoding an additional MYB transcription factor to produce a modified plant having increased expression of the exogenous gene encoding the additional MYB transcription factor, as well as increased expression of the exogenous gene encoding the TT2-type MYB transcription factor.

### EXAMPLE 1

### Materials and Methods

Chemicals. Cyanidin chloride, delphinidin chloride, pelargonidin chloride, (+)-catechin, (−)epicatechin, procyanidin B1 and procyanidin B2 standards were purchased from Sigma (Sigma, St. Louis, USA). All standards were dissolved in HPLC-grade methanol and stored at −20° C. until use.

Plant material and growth conditions. Seeds of G. raimondii (pI530900), G. arboreum (pI 529711) and G. hirsutum (pI636346) were ordered from USDA. Seeds were treated with concentrated sulfuric acid for 30 s before sterilizing using 70% ethanol and 20% bleach. Sterilized seeds were left on moisturized filter paper in petri dishes until germinated, and young seedlings were first kept in a growth chamber (25° C., 16 h light/8 h dark) for one month before eventually moving to the greenhouse.

Arabidopsis seeds were surface sterilized before germinating on ½ MS solid medium, and 2-week old seedlings were transferred to soil and kept in Conviron growth chambers under long day conditions (22° C., 16 h light/8 h dark; for genetic transformation) or short day conditions (22° C., 8 h light/16 h dark; for isolation of protoplasts).

Gene cloning and vector construction. Trifolium arvense TaMYB14 and Arabidopsis TT2 protein sequences were used to BLAST against the newly released cotton genome (cgp.genomics.org.cn) and top candidates were selected based on sequence similarity. Primers were designed based on the gene models CotAD_06578 and CotAD_18743.

RNA from cotton and Arabidopsis was extracted using PureLink® Plant RNA Reagent (Thermo Fisher, Tex., USA), treated with DNase and reverse-transcribed into cDNA using an iSCRIPT™ advanced cDNA synthesis kit (Bio-Rad, Calif., USA). PCR was performed using Phusion® High-Fidelity DNA polymerase (New England BioLabs, Mass., USA) at 1 cycle of 30 s at 98° C., 35 cycles of 10 s at 98° C., 30 s at 58° C. and 1 min at 72° C. followed by a final extension of 5 min at 72° C. All primers used in the present work are described in Table 1 below. PCR products were run on 1.2% agarose gels, and purified and ligated to pENTR™/D-TOPO® vector following the manufacturer's instructions. Gene sequences were verified by sequencing and subsequently ligated to the Gateway™ destination vector pB7WG2D for Arabidopsis and Medicago transformations and to pMDC43 vector for expression to determine subcellular localization.

TABLE 1

| Primer name | SEQ ID NO. | Sequence (5′-3′) | Purpose |
|---|---|---|---|
| GhMYB36F | 5 | caccATGGGAAGGAGTCCATGTTGC | Gene |
| | | | cloning |
| GhMYB36R | 6 | TCATGGCCATTCATCAGTGTCA | Gene |
| | | | cloning |
| GhMYB10F | 7 | caccATGGGAAGGAGTCCTTGTTGTT | Gene |
| | | | cloning |
| GhMYB10R | 8 | TCATTCAATTCCATTTTCAATAAATGGAG | Gene |
| | | | cloning |
| GhMYB36qRTF | 9 | TAGCAGCAAGGTGATGGTCCCA | qRT-PCR |
| GhMYB36qRTR | 10 | CCGCCATCACTGTACAATGACTC | qRT-PCR |
| GhMYB10qRTF | 11 | CACCACACACAAAACAGAGACCC | qRT-PCR |
| GhMYB10qRTR | 12 | TCCGATGATATATGATCGGTCGTCC | qRT-PCR |
| GhHisqRTF | 13 | CCGTACCAAGCAAACCGCCCG | qRT-PCR |
| GhHisqRTR | 14 | CCAGGACGGTATCGATGAGGCTT | qRT-PCR |
| EF1aF | 15 | AGGTCCACCAACCTTGACTG | RT-PCR |
| EF1aR | 16 | GAGACTCGTGGTGCATCTCA | RT-PCR |
| AtANRF | 17 | ATGGACCAGACTCTTACACACA | RT-PCR |
| AtANRR | 18 | TTATTTAGCTTTGATCAATCCTTTTGAC | RT-PCR |
| GhLAR1proF | 19 | caccCATCCCAATTTGAAATGATTGA | Promoter |
| | | | cloning |
| GhLAR1proR | 20 | GGTTAGCGTTATTGCAAGATTAGA | Promoter |
| | | | cloning |
| GrANR1proF | 21 | caccGTAGATGGAGGTGGAGGAGTGTG | Promoter |
| | | | cloning |
| GrANR1proR | 22 | GCTTCTGTCTTAATCTGTGTGTGTGTG | Promoter |
| | | | cloning |
| GrLAR1proF | 23 | caccCAACGACTTAGACATCCTAACTTAACAC | Promoter |
| | | | cloning |
| GrLAR1proR | 24 | GGTTAGCGTTATTGCAAGATTAGATAAAAC | Promoter |
| | | | cloning |
| MtANRqRTF | 25 | CAACTTCTGGTCGATACATTTGC | qRT-PCR |
| MtANRqRTR | 26 | CTGAGGGTATCGTTTGCTGAG | qRT-PCR |
| MtLARqRTF | 27 | CCGTTGGATCAATTGCACATC | qRT-PCR |
| MtLARqRTR | 28 | GTAACAGTTGGTAGAGGGTCG | qRT-PCR |
| MtDFRqRTF | 29 | GTTTCAAAGACACTTGCGGAG | qRT-PCR |
| MtDFRqRTR | 30 | GTGATAGGAGAAAGGGCAGTG | qRT-PCR |
| MtTUBqRTF | 31 | TTTGCTCCTCTTACATCCCGTG | qRT-PCR |
| MtTUBqRTR | 32 | CAGCACACATCATGTTTTTGG | qRT-PCR |

Transient expression in Arabidopsis protoplasts and tobacco leaves. Transient expression to study promoter-MYB protein interactions was performed according to Sheen et al (molbio.mgh.harvard.edu/sheenweb/). The Renilla luciferase gene was co-transfected as reference for normalization. Quantification of luciferase was performed using the Promega Dual-Luciferase Reporter Assay System (Promega, Wis., USA) in a BioTek Synergy MX Plate Reader. Subcellular localization of MYB-GFP fusion protein was studied by transient expression in Nicotiana benthamaia leaves according to protocols described earlier (Petrie et al. 2010). Visualization of GFP signal was examined with a Zeiss LSM 710 confocal microscope and processed using the Zeiss ZEN program.

Plant transformation and genotyping. Genetic transformation of Arabidopsis plants was based on the floral dip method (Clough and Bent 1998). Arabidopsis plant and Medicago hairy root transformations were performed as previously described (Liu et al. 2014). Transgenic hairy root lines were sub-cultured every 3 weeks. Genotyping of transformants was performed using Promega GoTaq Green Mastermix following the manufacturer's instructions.

qRT-PCR was performed using PowerUp™ SYBR® Green Master Mix (Thermo Fisher) and primers shown in Table 1 above according to the manufacturer's manual, and data were analyzed using QuantStudio 6 software.

PA and anthocyanin extraction and HPLC analysis. Extraction and quantification of PAs and anthocyanins were performed as described previously (Pang et al. 2008). DMACA (dimethylaminocinnamaldehyde) staining of Arabidopsis seeds was performed using dry seeds soaked in 1% DMACA solution (w/v, in 50% methanol +50% 12 N HCl) overnight followed by washing 3 times with 70% ethanol. Pictures of stained seeds were obtained with a Leica MZ10F microscope (Leica, Buffalo Grove, Ill.). Normal phase and reverse phase HPLC analyses were run on an Agilent HP1100 HPLC system as described previously (Liu et al. 2014).

Phylogenetic and statistical analyses. Multiple protein sequence alignment was carried out using the ClustalW program and phylogenetic trees were constructed using MEGA6.0 software (Tamura et al. 2013). Statistical analysis of data was performed using Student's t-test, and P<0.05 was accepted as significant between two groups.

### EXAMPLE 2

### Identification of TT2-Type MYB Transcription Factors in Cotton

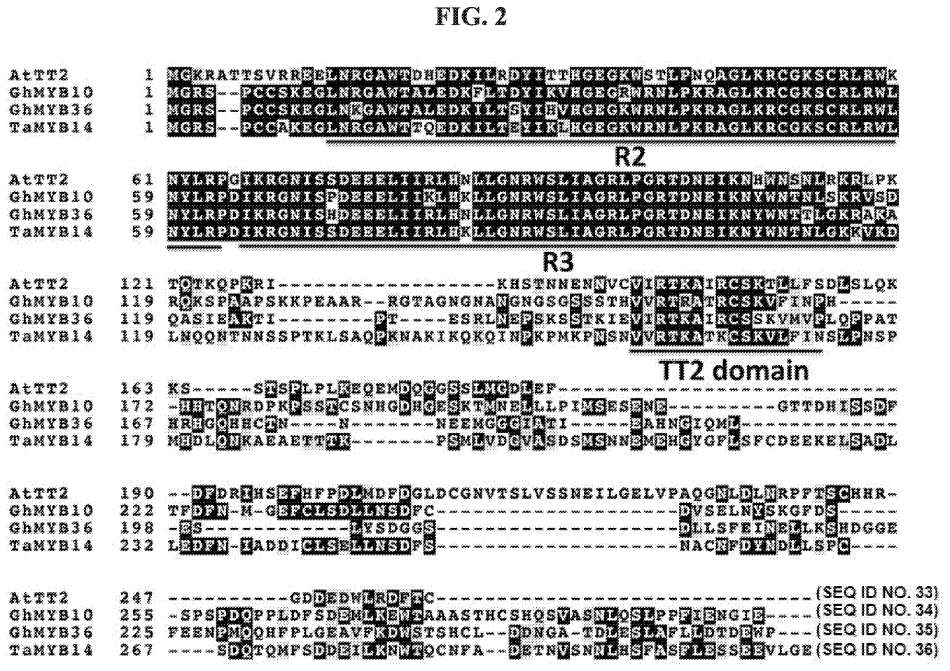

TT2-type MYB transcription factors belong to the R2R3-type MYB gene family and have been characterized in species such as Arabidopsis, Trifolium, Lotus, Medicago, poplar and cacao. FIG. 2 shows a multiple protein sequence alignment of MYB transcription factors. The GHMYB36, GHMYB10, AtTT2 and TaMYB14 protein sequences were aligned using ClustalW program, and conserved motifs are highlighted with a black line underneath. By performing BLAST search against the newly sequenced tetraploid cotton genome and analysis of the Cotton Gene Index 11 (Samuel Yang et al., 2016) using TaMYB14 and AtTT2, two candidate genes, GHMYB36 and GHMYB10, with high sequence similarities were found, indicating that multiple homologs of TT2 may exist in tetraploid cotton. These two genes were isolated from cotton leaf tissue using RT-PCR. Sequencing indicated that GHMYB36 encodes a protein of 271 amino acids and GHMYB10 encodes a 302-amino acid protein. Both genes have the conserved V[I/V]RT[K/R]A[I/T]RCS motif shared by other members in this family, as shown in FIG. 2.

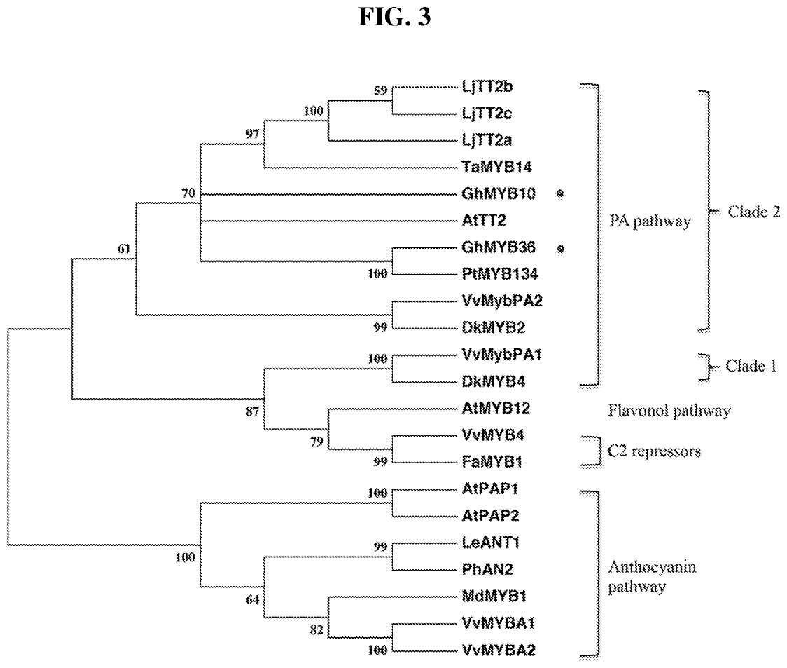

Further phylogenetic analysis with other known MYBs from different species and with different functions clearly placed both of these two GhMYBs into clade 2 containing genes that are related to the PA biosynthesis pathway. FIG. 3 shows a phylogenetic tree of GHMYB36, GHMYB10 and other known R2R3-type MYB transcription factors. The tree was constructed using MEGA6 software by the neighbor-joining method with 1000 bootstrap replicates. Numbers next to each node represent confidence percentages. Genbank accession numbers are LjTT2a (AB300033.2), LjTT2b (AB300034.2), LjTT2c (AB300035.1), TaMYB14 (JN049641.1), PtMYB134 (FJ573151.1), PhAN2 (AF146702.1), MdMYB1 (DQ886414.1), AtTT2 (NP_198405.1), VvMybPA1 (AM259485.1), VvMybPA2 (EU919682.1), DkMYB2 (AB503699.1), DkMYB4 (AB503701.1), AtMYB12 (DQ224277.1), VvMYB4 (EF113078.1), FaMYB1 (AF401220.1), AtPAP1 (DQ222406.1), AtPAP2 (NP_176813.1), LeANT1 (AY348870.1), VvMYBA1 (AB097923.1), VvMYBA2 (AB097924.1).

GHMYB36 is 99% (812/816 of nucleotides, 268/271 of amino acids) similar to gene model Cotton_A_05641 in G. arboreum, and GHMYB10 is 99% similar (899/909 nt, 298/302 aa) to gene model Gorai.010G087200 in G. raimondii. Both genes were annotated as TT2-type MYBs. It is therefore reasonable to assume that tetraploid cotton harbors both genes originating from each ancestor during hybridization.

### EXAMPLE 3

### Expression Profiles of GHMYB36 and GHMYB10

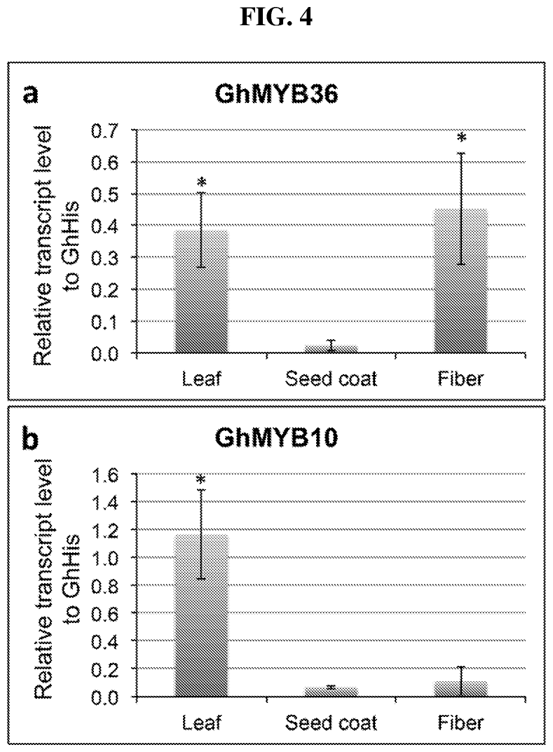

To better understand the expression profiles of GHMYB36 and GHMYB10, RT-PCR analysis was performed on RNA extracted from various tissues, using primers shown in Table 1. FIG. 4 shows transcript levels of GHMYB36 (a) and GHMYB10 (b) in different tissues of cotton as determined by qRT-PCR, with n=3 and *, p<0.05. The results showed that GHMYB36 had slightly higher transcript levels in developing fibers than in leaf, while GHMYB10 had highest transcript levels in leaf, and expression of both genes could also be detected in the seed coat. This is consistent with the observation that cotton accumulates high amounts of PAs in leaves. In Lotus japonicus, different tissue-specific expression patterns were found for its three homologs of AtTT2, similar to the present observation with GHMYB36 and GHMYB10.

To study the subcellular localization of the cotton MYB proteins, GFP-tagged GHMYB36 and GHMYB10 were transiently expressed in tobacco leaves by Agrobacterium-mediated infiltration. GFP-GhMYB36 and GFP-GhMYB10 fusion proteins were transiently expressed in N. benthamiana leaves and visualized by laser confocal microscopy. By scanning the GFP signal it was found that both fusion proteins localized to the cell nucleus, as expected for transcription factors.

### EXAMPLE 4

### GHMYB36 and GHMYB10 Activte Promotors of Cotton LAR and ANR as Part of an MBW Complex

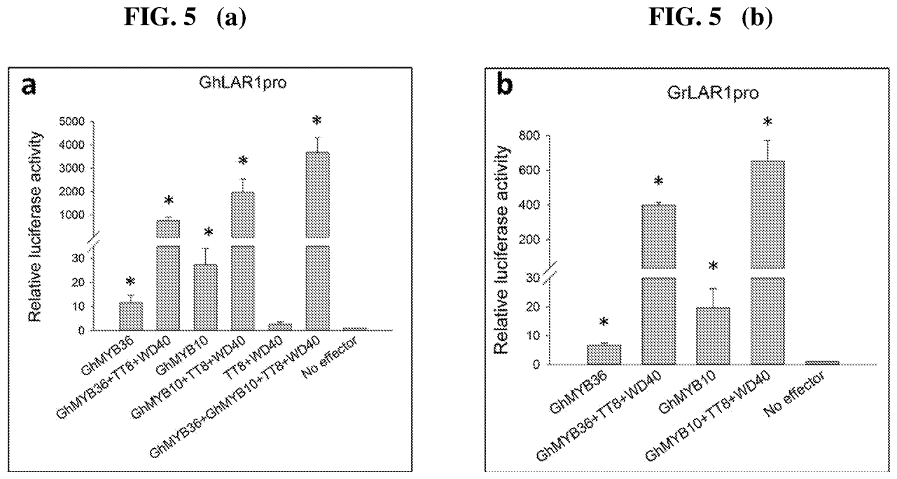

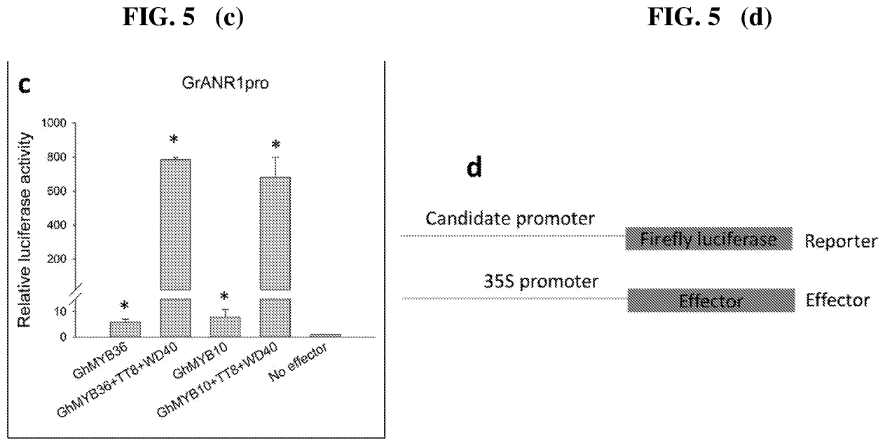

TT2 and other homologs can recruit other transcription factors and bind to promoter regions of structural genes such as ANR or LAR. To determine whether the same is true for GHMYB36 and GHMYB10, the LAR1 promoter was targeted for isolation from G. hirsutum. Because attempts to isolate the GhANR promoter failed, promoters of LAR and ANR genes were instead isolated from G. raimondii and G. arboreum. Sequence alignment of the ANR promoters from tetraploid and both diploid cottons showed that similarity was high, and they all contain the proposed MYB binding sites. FIG. 5 shows transactivation assays using GHMYB36 and GHMYB10 with GhLAR (a), GrLAR (b) and GrANR (c) promoters. Numbers on y-axis represent relative expression levels of firefly luciferase gene normalized to control groups that were transformed with promoters only. Renilla luciferase gene was used as reference to check transformation efficiency. Constructs used in this assay are indicated in panel (d), with n=3 and * indicating p<0.05. Results from transactivation assays showed that expression of GHMYB36 or GHMYB10 individually slightly activated the promoters of cotton LARs and ANR, with activation levels 10-20 fold higher than in cells transfected with promoter DNA only.

Previous studies showed that Medicago truncatula MtWD40 and MtTT8 (bHLH) are part of the MYB-bHLH-WD40 complex regulating PA biosynthesis, confirmed by transactivation assays using MtMYB14 and MtMYB5. The ability of GHMYB36 and GHMYB10 to activate promoters in the presence of co-transfected MtWD40 and MtTT8 was therefore determined. For the GhLAR1 promoter, expressing MtWD40 and MtTT8 without GHMYB36 or GHMYB10 gave activation levels similar to the control with promoter DNA only. When combining MtWD40 and MtTT8 with either GHMYB36 or GHMYB10, the activation levels were increased about 780- and 2000-fold, suggesting the involvement of a ternary complex between GHMYB36 or GHMYB10 with MtWD40 and MtTT8 to strongly enhance activation of the cotton LAR promoter. Interestingly, when combining both GHMYB36 and GHMYB10 along with MtWD40 and MtTT8, the activation level reached up to 3800 fold, about 30% more than with GHMYB36 and GHMYB10 individually. Similar results were obtained using LAR1 and ANR promoters from diploid cotton, or even with the M. truncatula MtANR and MtLAR promoters. It is therefore clear that both GHMYB36 and GHMYB10 function as part of a MBW complex that can operate with components from both cotton and non-closely related species, as Medicago is a legume.

There are two candidate LAR genes annotated in both G. arboreum (Cotton_A_34464, GaLAR1 and Cotton_A_01395, GaLAR2) and G. raimondii (Gorai008G186500, GrLAR1 and Gorai008G285400, GrLAR2). Transactivation assays using Trifolium arvense TaMYB14, which is another homolog of AtTT2, together with MtWD40 and MtTT8, showed that the activation level was significantly higher for one LAR (about 600-fold for GaLAR1, and 680-fold GrLAR1) than the other (about 30-fold for GaLAR2 and 20-fold for GrLAR2). Similar results were observed on replacing TaMYB14 with MtMYB14. It therefore appears that ANR and LAR1, but not LAR2, are the main targets of MYB regulators in cotton.

### EXAMPLE 5

### Both GHMYB36 and GHMYB10 can Complement the Arabidopsis TT2 Mutant

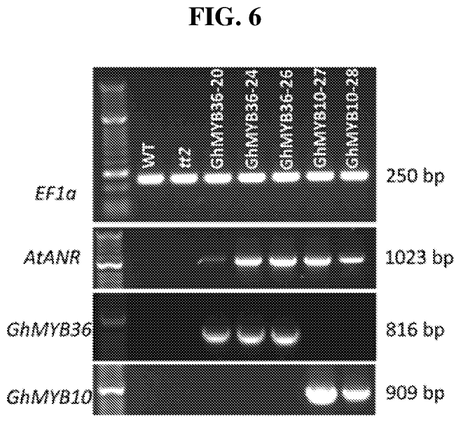

The tt2 knockout mutant of Arabidopsis shows a transparent testa phenotype due to repressed expression of AtANR and other genes in the PA biosynthesis pathway. To test whether GHMYB36 and GHMYB10 are true functional orthologs of AtTT2, GHMYB36 and GHMYB10 were ectopically expressed in the Arabidopsis tt2 mutant. Among over 10 transgenic lines tested from each experiment (including transgenic lines GHMYB36-24, GHMYB36-26, GHMYB10-27 and GHMYB10-28), PA accumulation was restored in the seed coats, although at various levels. RT-PCR analysis using tissues from two-week old Arabidopsis seedlings showed transgene expression as well as AtANR expression in vegetative tissues, indicating that GHMYB36 and GHMYB10 are able to induce AtANR expression in planta. FIG. 6 shows RT-PCR of Arabidopsis housekeeping gene EF1a, endogenous AtANR, GHMYB36 and GHMYB10 using leaf samples from wild type, tt2 and transgenic lines GHMYB36-20, GHMYB36-24, GHMYB36-26, GHMYB10-27 and GHMYB10-28. Numbers on the right represent sizes of PCR products. Roots of some transgenic lines overexpres sing GHMYB36 exhibited a distinct purple color when stained with 1% DMACA, indicating high levels of PA accumulation. No PAs were detected in leaf tissues based on DMACA staining results. Together the data show that both GHMYB36 and GHMYB10 are able to complement the tt2 mutation, although lines expressing GHMYB36 showed the better ability to accumulate PAs.

### EXAMPLE 6

### GHMYB36 and GHMYB10 can Induc PA Accumulation in Medicago Hairy Roots

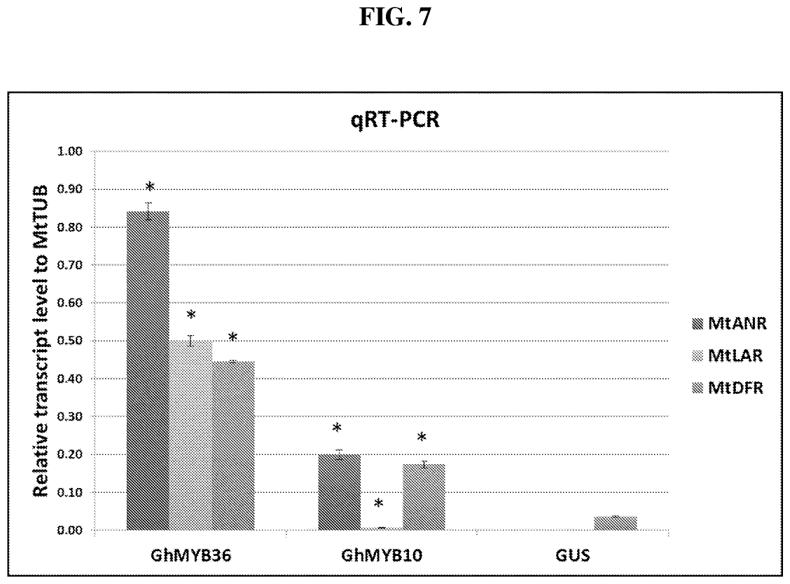

MtMYB14 and AtTT2 have been shown to induce PA accumulation in Medicago hairy root cultures. To test if GHMYB36 and GHMYB10 have similar activities, 35S::GHMYB36 and 35S::GHMYB10 constructs were transformed for constitutive expression into M. truncatula hairy roots, with 35S::GUS as negative control. DMACA staining clearly showed increased PA concentrations in GHMYB36 and GHMYB10 transgenic lines, as indicated by a dark blue-green color after ethanol washes, compared to the yellow color in the GUS lines. qRT-PCR analysis showed that transcript levels of key enzymes in PA biosynthesis, including MtANR, MtLAR and MtDFR, were all significantly higher in GHMYB36 and GHMYB10 expressing lines compared to GUS expressing lines. FIG. 7 shows transcript levels of MtANR, MtLAR and MtDFR in transgenic Medicago hairy root cultures transformed with GUS, GHMYB36 or GHMYB10. Numbers on y-axis represent relative expression levels normalized to MtTUB gene, where n=4 and * indicates p<0.05. These results further confirm the transactivation assay results, indicating that ANR and LAR are target genes of GHMYB36 and GHMYB10.

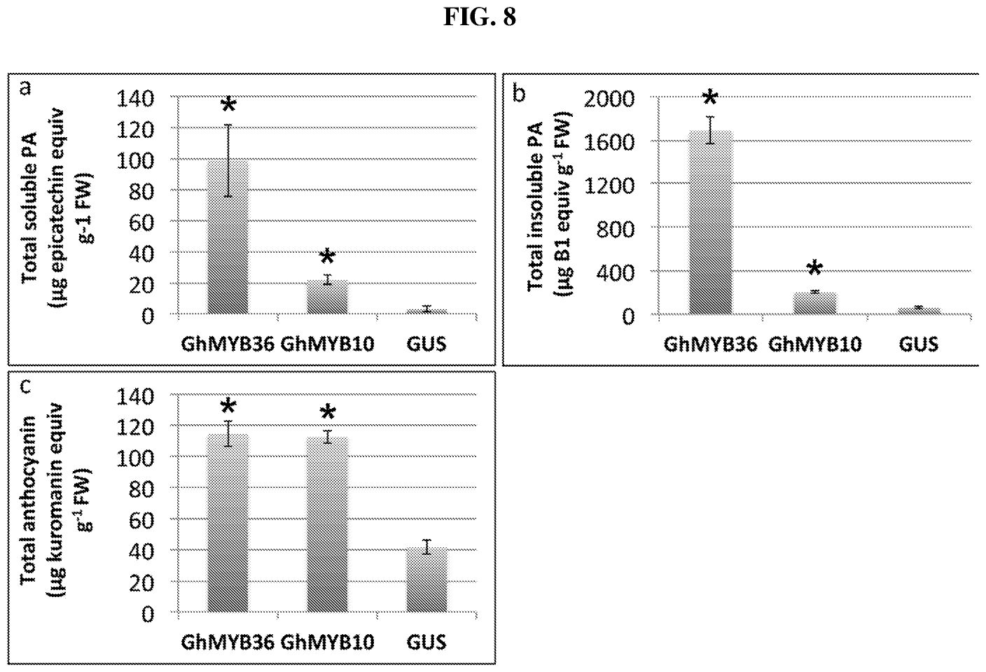

FIG. 8 shows quantification of soluble and insoluble PAs and anthocyanins ((a) epicatechin, (b) insoluble PA, and (c) anthocyanin) in transgenic Medicago hairy root cultures transformed with GUS, GHMYB36 or GHMYB10, where n=3 and * indicates p<0.05. Levels of both soluble and insoluble PAs were significantly higher in hairy roots expressing GHMYB36 or GHMYB10 compared to GUS control lines, although GHMYB36 lines had much higher concentrations of PAs than GHMYB10 lines. Anthocyanin levels were also higher in GHMYB36 and GHMYB10 expressing lines than in GUS expressing lines, similar to results of earlier experiments analyzing heterologous expression of AtTT2.

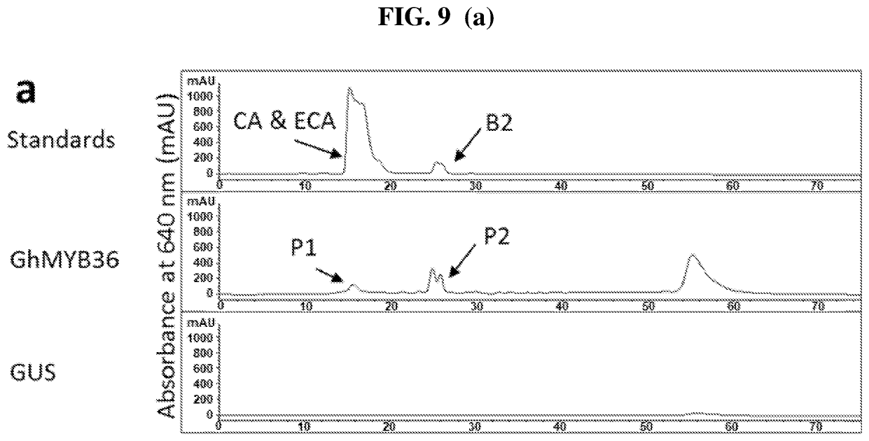

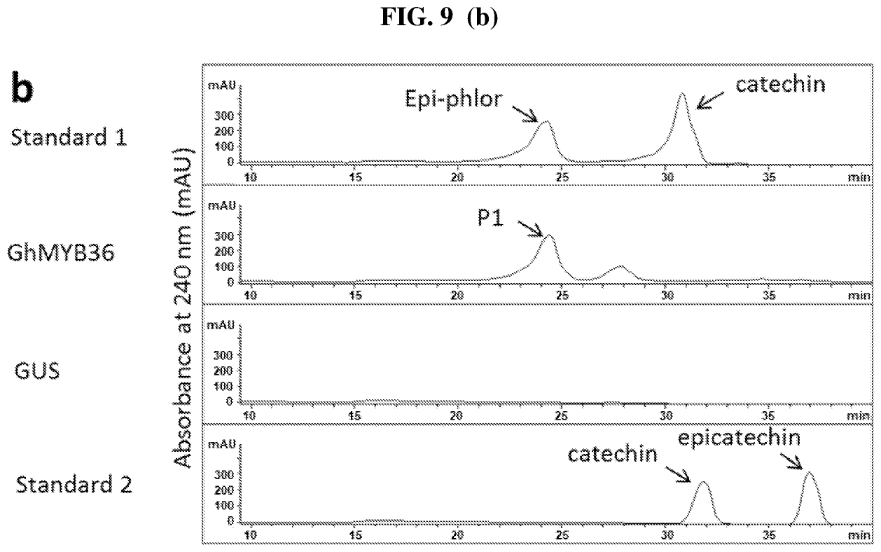

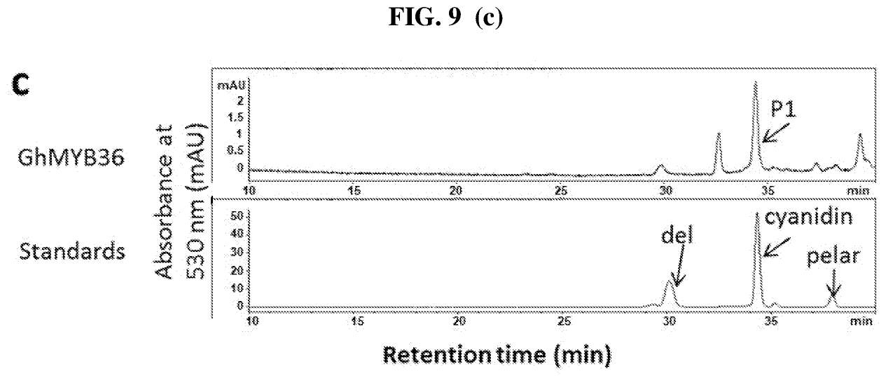

Arabidopsis and Medicago accumulate epicatechin-based PA polymers, whereas cotton contains more gallocatechin- and catechin-based PAs. The PAs produced through activation of the pathway in Medicago hairy roots by GHMYB36 were therefore analyzed to determine their size and composition. First the soluble PAs in GHMYB36 lines were analyzed by normal phase high performance liquid chromatography (HPLC) followed by post-column derivatization with DMACA (Peel and Dixon 2007). FIG. 9 shows analysis of PA size and composition in M. truncatula hairy root transformants, where (a) shows normal phase HPLC with post-column derivatization showing size distribution of soluble PAs from Medicago hairy root cultures transformed with GUS or GHMYB36, with the standards catechin, epicatechin and procyanidin B2. In addition to some PA dimers, the major proportion of the PAs consisted of polymers with mean degree of polymerization (average number of monomeric flavan 3-ols in polymers) of about 10. Next, phloroglucinolysis was performed on the soluble portion of the PAs extracted from the Medicago roots, and the products were compared to analytical standards. FIG. 9(b) shows HPLC analysis of phloroglucinolysis products of PAs from Medicago hairy root cultures transformed with GHMYB36 or GUS, where standard 1 was procyanidin B1 control and standard 2 was a mixture of catechin and epicatechin. Results showed that the soluble PAs in transgenic lines were still mainly epicatechin-based, as revealed by the presence of predominant epicatechin-phloroglucinol extension units.

Lastly, acid hydrolysis was performed with insoluble PAs extracted from both GHMYB36 lines, and the products were analyzed on reverse phase HPLC. FIG. 9(c) shows acid butanol hydrolysis of insoluble PAs extracted from GHMYB36 lines. Standards used were cyanidin chloride, delphinidin chloride and pelargonidin chloride. Results showed that cyanidin was the major unit released, indicating that the insoluble PAs were comprised mainly of (epi)-catechin units.

### REFERENCES CITED

The following documents and publications are hereby incorporated by reference.

### Non-Patent Publications

Akagi T, Ikegami A, Tsujimoto T, Kobayashi S, Sato A, Kono A, Yonemori K (2009) DkMyb4 is a Myb transcription factor involved in proanthocyanidin biosynthesis in persimmon fruit. Plant Physiology 151: 2028-2045

Albert S, Delseny M, Devic M (1997) BANYULS, a novel negative regulator of flavonoid biosynthesis in the Arabidopsis seed coat. Plant Journal 11: 289-299

Aron P M, Kennedy J A (2008) Flavan-3-ols: Nature, occurrence and biological activity. Molecular Nutrition & Food Research 52: 79-104

Bell A A, El-Zik K M, Thaxton P M (1992) Chemistry, biological significance, and genetic control of proanthocyanidins in cotton (Gossypium spp.). Plant Polyphenols. Springer, pp 571-595

Bogs J, Jaffe F W, Takos A M, Walker A R, Robinson S P (2007) The grapevine transcription factor VvMYBPA1 regulates proanthocyanidin synthesis during fruit development. Plant Physiology 143: 1347-1361

Cavallini E, Matus J T, Finezzo L, Zenoni S, Loyola R, Guzzo F, Schlechter R, Ageorges A, Arce-Johnson P, Tornielli G B (2015) The phenylpropanoid pathway is controlled at different branches by a set of R2R3-MYB C2 repressors in grapevine. Plant Physiology 167: 1448-1470

Clough S J, Bent A F (1998) Floral dip: a simplified method for Agrobacterium-mediated transformation of Arabidopsis thaliana. Plant Journal 16: 735-743

de Colmenares N G, Ramírez-Martinez J R, Aldana J O, Ramos-Niño M E, Clifford M N, Pékerar S, Méndez B (1998) Isolation, characterisation and determination of biological activity of coffee proanthocyanidins. Journal of the Science of Food and Agriculture 77: 368-372

Debeaujon I, Nesi N, Perez P, Devic M, Grandjean O, Caboche M, Lepiniec L (2003) Proanthocyanidin-accumulating cells in Arabidopsis testa: regulation of differentiation and role in seed development. Plant Cell 15: 2514-2531

Dixon R A, Liu C, Jun J H (2013) Metabolic engineering of anthocyanins and condensed tannins in plants. Current Opinion in Biotechnology 24: 329-335

Dixon R A, Xie D-Y, Sharma S B (2005) Proanthocyanidins—a final frontier in flavonoid research? New Phytologist 165: 9-28

Feng H, Li Y, Wang S, Zhang L, Liu Y, Xue F, Sun Y, Wang Y, Sun J (2014) Molecular analysis of proanthocyanidins related to pigmentation in brown cotton fibre (Gossypium hirsutum L.). Journal of Experimental Botany 65: 5759-5769

Feng H, Tian X, Liu Y, Li Y, Zhang X, Jones B J, Sun Y, Sun J (2013) Analysis of flavonoids and the flavonoid structural genes in brown fiber of upland cotton. PLoS One 8: e58820

Gonzalez A, Mendenhall J, Huo Y, Lloyd A (2009) TTG1 complex MYBs, MYB5 and TT2, control outer seed coat differentiation. Developmental Biology 325: 412-421

Hancock K R, Collette V, Fraser K, Greig M, Xue H, Richardson K, Jones C, Rasmussen S (2012) Expression of the R2R3-MYB transcription factor TaMYB14 from Trifolium arvense activates proanthocyanidin biosynthesis in the legumes Trifolium repens and Medicago sativa. Plant Physiology 159: 1204-1220

Hinchliffe D J, Condon B D, Thyssen G, Naoumkina M, Madison C A, Reynolds M, Delhom C D, Fang D D, Li P, McCarty J (2016) The GhTT2_A07 gene is linked to the brown colour and natural flame retardancy phenotypes of Lc1 cotton (Gossypium hirsutum L.) fibres. Journal of Experimental Botany: erw312

Hovav R, Faigenboim-Doron A, Kadmon N, Hu G, Zhang X, Gallagher J P, Wendel J F (2015) A transcriptome profile for developing seed of polyploid cotton. Plant Genome 8

Ito C, Oki T, Yoshida T, Nanba F, Yamada K, Toda T (2013) Characterisation of proanthocyanidins from black soybeans: Isolation and characterisation of proanthocyanidin oligomers from black soybean seed coats. Food Chemistry 141: 2507-2512

Jun J H, Liu C, Xiao X, Dixon R A (2015) The transcriptional repressor MYB2 regulates both spatial and temporal patterns of proanthocyandin and anthocyanin pigmentation in Medicago truncatula. Plant Cell 27: 2860-2879

Kovinich N, Saleem A, Arnason J T, Miki B (2012) Identification of two anthocyanidin reductase genes and three red-brown soybean accessions with reduced anthocyanidin reductase 1 mRNA, activity, and seed coat proanthocyanidin amounts. Journal of Agricultural and Food Chemistry 60: 574-584

Li F, Fan G, Lu C, Xiao G, Zou C, Kohel R J, Ma Z, Shang H, Ma X, Wu J, Liang X, Huang G, Percy R G, Liu K, Yang W, Chen W, Du X, Shi C, Yuan Y, Ye W, Liu X, Zhang X, Liu W, Wei H, Wei S, Huang G, Zhang X, Zhu S, Zhang H, Sun F, Wang X, Liang J, Wang J, He Q, Huang L, Wang J, Cui J, Song G, Wang K, Xu X, Yu JZ, Zhu Y, Yu S (2015) Genome sequence of cultivated Upland cotton (Gossypium hirsutum TM-1) provides insights into genome evolution. Nature Biotechnology 33: 524-530

Li H, Flachowsky H, Fischer T C, Hanke M V, Forkmann G, Treutter D, Schwab W, Hoffmann T, Szankowski I (2007) Maize Lc transcription factor enhances biosynthesis of anthocyanins, distinct proanthocyanidins and phenylpropanoids in apple (Malus domestica Borkh.). Planta 226: 1243-1254

Li T C, Fan H H, Li Z P, Wei J, Lin Y, Cai Y P (2012) The accumulation of pigment in fiber related to proanthocyanidins synthesis for brown cotton. Acta Physiologiae Plantarum 34: 813-818

Liu C, Jun J H, Dixon R A (2014) MYBS and MYB14 play pivotal roles in seed coat polymer biosynthesis in Medicago truncatula. Plant Physiology 165: 1424-1439

Liu Y, Shi Z, Maximova S, Payne M J, Guiltinan M J (2013) Proanthocyanidin synthesis in Theobroma cacao: genes encoding anthocyanidin synthase, anthocyanidin reductase, and leucoanthocyanidin reductase. BMC Plant Biology 13: 1-19

Liu, C., Shulaev, V. and Dixon, R. A. (2016) Role of leucoanthocyanidin reductase in extension of proanthocyanidins in Medicago. Nature Plants, in press.

Mellway R D, Constabel C P (2009) Metabolic engineering and potential functions of proanthocyanidins in poplar. Plant Signaling & Behavior 4: 790-792

Nesi N, Jond C, Debeaujon I, Caboche M, Lepiniec L (2001) The Arabidopsis TT2 gene encodes an R2R3 MYB domain protein that acts as a key determinant for proanthocyanidin accumulation in developing seed. Plant Cell 13: 2099-2114

Pang Y, Abeysinghe I S B, He J, He X, Huhman D, Mewan K M, Sumner L W, Yun J, Dixon R A (2013) Functional characterization of proanthocyanidin pathway enzymes from tea and their application for metabolic engineering. Plant Physiology 161: 1103-1116

Pang Y, Peel G J, Sharma S B, Tang Y, Dixon R A (2008) A transcript profiling approach reveals an epicatechin-specific glucosyltransferase expressed in the seed coat of Medicago truncatula. Proceedings of the National Academy of Sciences USA 105: 14210-14215

Pang Y, Peel G J, Wright E, Wang Z, Dixon R A (2007) Early steps in proanthocyanidin biosynthesis in the model legume Medicago truncatula. Plant Physiology 145: 601-615

Peel G J, Dixon R A (2007) Detection and quantification of engineered proanthocyanidins in transgenic plants. Natural Product Communications 2: 1009-1014

Peel G J, Pang Y, Modolo L V, Dixon R A (2009) The LAP1 MYB transcription factor orchestrates anthocyanidin biosynthesis and glycosylation in Medicago. Plant Journal 59: 136-149

Peters D J, Constabel C P (2002) Molecular analysis of herbivore-induced condensed tannin synthesis: cloning and expression of dihydroflavonol reductase from trembling aspen (Populus tremuloides). Plant Journal 32: 701-712

Petrie J R, Shrestha P, Liu Q, Mansour M P, Wood C C, Zhou X R, Nichols P D, Green A G, Singh S P (2010) Rapid expression of transgenes driven by seed-specific constructs in leaf tissue: DHA production. Plant Methods 6: 8

Prasad R, Vaid M, Katiyar S K (2012) Grape proanthocyanidins inhibit pancreatic cancer cell growth in vitro and in vivo through induction of apoptosis and by targeting the PI3K/Akt pathway. PLoS ONE 7: e43064

Robbins M P, Paolocci F, Hughes J W, Turchetti V, Allison G, Arcioni S, Morris P, Damiani F (2003) Sn, a maize bHLH gene, modulates anthocyanin and condensed tannin pathways in Lotus corniculatus. Journal of Experimental Botany 54: 239-248

Samuel Yang S, Cheung F, Lee J J, Ha M, Wei N E, Sze S H, Stelly D M, Thaxton P, Triplett B, Town C D (2006) Accumulation of genome-specific transcripts, transcription factors and phytohormonal regulators during early stages of fiber cell development in allotetraploid cotton. Plant Journal 47: 761-775

Tamura K, Stecher G, Peterson D, Filipski A, Kumar S (2013) MEGA6: Molecular Evolutionary Genetics Analysis Version 6.0. Molecular Biology and Evolution 30: 2725-2729

Terrier N, Torregrosa L, Ageorges A, Vialet S, Verriès C, Cheynier V, Romieu C (2009) Ectopic expression of VvMybPA2 promotes proanthocyanidin biosynthesis in grapevine and suggests additional targets in the pathway. Plant Physiology 149: 1028-1041

Verpoorte R (2000) Secondary metabolism. Metabolic engineering of plant secondary metabolism. Springer, pp 1-29

Wu K-M, Lu Y-H, Feng H-Q, Jiang Y-Y, Zhao J-Z (2008) Suppression of cotton bollworm in multiple crops in China in areas with Bt toxin-containing cotton. Science 321: 1676-1678

Xiao Y H, Yan Q, Ding H, Luo M, Hou L, Zhang M, Yao D, Liu H S, Li X, Zhao J, Pei Y (2014) Transcriptome and biochemical analyses revealed a detailed proanthocyanidin biosynthesis pathway in brown cotton fiber. PLoS One 9: e86344

Xie D-Y, Sharma S B, Dixon R A (2004) Anthocyanidin reductases from Medicago truncatula and Arabidopsis thaliana. Archives of Biochemistry and Biophysics 422: 91-102

Xie D-Y, Sharma S B, Paiva N L, Ferreira D, Dixon R A (2003) Role of anthocyanidin reductase, encoded by BANYULS in plant flavonoid biosynthesis. Science 299: 396-399

Xie D Y, Sharma S B, Wright E, Wang Z Y, Dixon R A (2006) Metabolic engineering of proanthocyanidins through co-expression of anthocyanidin reductase and the PAP1 MYB transcription factor. Plant Journal 45: 895-907

Xu W, Lepiniec L, Dubos C (2014) New insights toward the transcriptional engineering of proanthocyanidin biosynthesis. Plant Signaling & Behavior 9: e28736

Yoshida K, Iwasaka R, Kaneko T, Sato S, Tabata S, Sakuta M (2008) Functional differentiation of Lotus japonicus TT2s, R2R3-MYB transcription factors comprising a multigene family. Plant and Cell Physiology 49: 157-169

Yu F, Barry T, Moughan P, Wilson G (1993) Condensed tannin and gossypol concentrations in cottonseed and in processed cottonseed meal. Journal of the Science of Food and Agriculture 63: 7-15

Zhao J, Dixon R A (2009) MATE transporters facilitate vacuolar uptake of epicatechin 3″-O-glucoside for proanthocyanidin biosynthesis in Medicago truncatula and Arabidopsis. Plant Cell 21: 2323-2340

Zhu Y, Wang H, Peng Q, Tang Y, Xia G, Wu J, Xie D-Y (2015) Functional characterization of an anthocyanidin reductase gene from the fibers of upland cotton (Gossypium hirsutum). Planta 241 : 1075-1089

What is claimed is:

1. A method for producing a modified plant having increased proanthocyanidin (PA) content in cells of the plant compared to an unmodified plant of the same species, comprising:
increasing expression of at least one gene encoding a TT2-type MYB transcription factor in the cells of the plant; and
producing a modified plant having increased expression of TT2-type MYB transcription factors and increased proanthocyanidin (PA) content in cells of the modified plant.

2. The method of claim 1, wherein the gene encoding a TT2-type MYB transcription factor is a homolog of AtTT2 of Arabidopsis thaliana.

3. The method of claim 1, wherein the plant is a cotton plant and the gene encoding a TT2-type MYB transcription factor is GHMYB36 or GHMYB10.

4. The method of claim 3, wherein GHMYB36 comprises SEQ ID NO:1, and wherein GHMYB10 comprises SEQ ID NO:2.

5. The method of claim 1, wherein the plant is a soybean plant and the gene encoding a TT2-type MYB transcription factor is GmTT2A or GmTT2B.

6. The method of claim 5, wherein GmTT2A comprises SEQ ID NO:3, and wherein GmTT2B comprises SEQ ID NO:4.

7. The method of claim 1, wherein the step of increasing expression of the at least one gene encoding a TT2-type MYB transcription factor comprises introducing a mutation into the gene encoding a TT2-type MYB transcription factor in cells of the plant, wherein the mutation results in increased expression of the gene encoding the TT2-type MYB transcription factor.

8. The method of claim 1, wherein the gene encoding a TT2-type MYB transcription factor is an exogenous gene, and the step of increasing expression of the at least one gene encoding a TT2-type MYB transcription factor comprises transforming at least one cell of the plant with the gene encoding a TT2-type MYB transcription factor to produce a modified plant having increased expression of the gene encoding the TT2-type MYB transcription factor.

9. The method of claim 8, wherein the plant is a non-cotton plant and the gene encoding TT2-type MYB transcription factors is GHMYB36 or GHMYB10.

10. The method of claim 9, wherein GHMYB36 comprises SEQ ID NO:1, and wherein GHMYB10 comprises SEQ ID NO:2.

11. The method of claim 8, wherein the plant is a non-soybean plant and the gene encoding a TT2-type MYB transcription factor is GmTT2A or GmTT2B.

12. The method of claim 11, wherein GmTT2A comprises SEQ ID NO:3, and wherein GmTT2B comprises SEQ ID NO:4.

13. The method of claim 8, wherein the plant is a Medicago truncatula or Arabidopsis thaliana plant.

14. The method of claim 8, wherein the plant is a cotton plant, a soybean plant, or an alfalfa plant.

15. The method of claim 8, further comprising the step of transforming at least one cell of the plant with a gene encoding an additional MYB transcription factor to produce a modified plant having increased expression of the gene encoding the additional MYB transcription factor.

16. A modified plant having increased proanthocyanidin (PA) content in cells of the plant compared to an unmodified plant of the same species, wherein cells of the plant have increased expression of at least one gene encoding a TT2-type MYB transcription factor.

17. A seed of the modified plant of claim 16.

18. The modified plant of claim 16, wherein the gene encoding a TT2-type MYB transcription factor is a homolog of AtTT2 of Arabidopsis thaliana.

19. The modified plant of claim 16, wherein the plant is a cotton plant and the gene encoding a TT2-type MYB transcription factors is GHMYB36 or GHMYB10.

20. The modified plant of claim 19, wherein GHMYB36 comprises SEQ ID NO:1, and wherein GHMYB10 comprises SEQ ID NO:2.

21. The modified plant of claim 16, wherein the plant is a soybean plant and the gene encoding a TT2-type MYB transcription factor is GmTT2A or GmTT2B.

22. The modified plant of claim 21, wherein GmTT2A comprises SEQ ID NO:3, and wherein GmTT2B comprises SEQ ID NO:4.

23. The modified plant of claim 16, wherein cells of the plant comprise a mutation in the gene encoding a TT2-type MYB transcription factor, and wherein the mutation results in increased expression of the gene encoding the TT2-type MYB transcription factor.

24. The modified plant of claim 16, wherein the gene encoding a TT2-type MYB transcription factor is an exogenous gene, and wherein at least one cell of the plant is transformed with the gene encoding a TT2-type MYB transcription factor to produce a modified plant having increased expression of the gene encoding the TT2-type MYB transcription factor.

25. The modified plant of claim 24, wherein the plant is a non-cotton plant and the gene encoding TT2-type MYB transcription factors is GHMYB36 or GHMYB10.

26. The modified plant of claim 25, wherein GHMYB36 comprises SEQ ID NO:1, and wherein GHMYB10 comprises SEQ ID NO:2.

27. The modified plant of claim 24, wherein the plant is a non-soybean plant and the gene encoding a TT2-type MYB transcription factor is GmTT2A or GmTT2B.

28. The modified plant of claim 27, wherein GmTT2A comprises SEQ ID NO:3, and wherein GmTT2B comprises SEQ ID NO:4.

29. The modified plant of claim 24, wherein the plant is a Medicago truncatula or Arabidopsis thaliana plant.

30. The modified plant of claim 24, wherein the plant is a cotton plant.

31. The modified plant of claim 24, wherein at least one cell of the plant is further transformed with a gene encoding an additional MYB transcription factor to produce a modified plant having increased expression of the gene encoding the additional MYB transcription factor.
